(19) United States
(12) Patent Application Publication
(10) Pub. No.: US 2011/0091938 A1
(43) Pub. Date: Apr. 21, 2011

(54) Starch Hydrolysis

(75) Inventors: Lin Wang, Iowa City, IA (US); Jeff M. Underwood, Muscatine, IA (US); Brian Peters, Muscatine, IA (US); Katherine Lauren Gregory, Muscatine, IA (US); Kevin Lester, Muscatine, IA (US)

(73) Assignee: Grain Processing Corporation, Muscatine, IA (US)

(21) Appl. No.: 12/908,286

(22) Filed: Oct. 20, 2010

Related U.S. Application Data
(60) Provisional application No. 61/253,357, filed on Oct. 20, 2009.

(51) Int. Cl. C12P 19/00

(52) U.S. Cl. 435/72

(57) ABSTRACT

Disclosed is a method for hydrolyzing starch. The starch is subjected to hydrolysis in the presence of a neutral calcium salt, the hydrolysis being conducted at a pH ranging from about 5.0 to about 5.5. The calcium salt may be a non-toxic and label-friendly salt such as calcium sulfate, calcium chloride or calcium phosphate, preferably naturally existing salts. The enzymatically catalyzed hydrolysis may be quenched using an organic acid, such as citric acid, preferably an acid that exists in nature, or with heat. Certain processing and product quality advantages may be realized via the disclosed method. The method may provide a variety of hydrolyzed products, including maltodextrins, syrup solids, and the like.

This application claims the benefit of U.S. Provisional Application No. 61/253,357, filed Oct. 20, 2009, the entire disclosure of which is hereby incorporated by reference.

### TECHNICAL FIELD

The invention is in the field of starch hydrolysis.

### BACKGROUND

Hydrolysis of starch to yield maltodextrins, oligosaccharides, and syrup solids has long been known in the art. A wide variety of starch hydrolysis products may be provided, these including maltodextrins (carbohydrate mixtures having a dextrose equivalent value of less than 20) and syrup solids (carbohydrate mixtures having a dextrose equivalent value greater than or equal to 20). These products are commercially valuable. Additionally, from these hydrolyzed starch products may be derived a wide variety of other commercially valuable materials, including such materials as hydrogenated maltodextrins, maltose syrups, and other products. Exemplary teachings concerning maltodextrins and related materials can be found in U.S. Pat. Nos. 7,405,293; 7,091,335; 6,919,446; 6,720,418; 6,613,898; 6,380,379; 4,782,143; and 4,603,110, all patents of Grain Processing Corporation of Muscatine, Iowa.

Starch typically is hydrolyzed under acidic conditions. In some instances, strong acids are used alone, without enzymatic catalysis. In many cases, however, a bacterially or otherwise derived alpha-amylase is used to catalyze the hydrolysis. Enzymatic catalysis can improve the efficiency of the hydrolysis and in some cases can help to achieve a desirable carbohydrate molecular weight profile. The enzymes employed can include glycoside hydrolases, typically glucoamylases and alpha amylases. Such enzymes can be obtained from any suitable source, including bacteria, plants, or fungi.

In a typical enzymatically catalyzed reaction, primary liquefaction occurs at an elevated temperature of approximately 105-108° C. in a jet-cooking step. For the enzymes to survive this high temperature, calcium addition often is required. Calcium is believed to fortify the enzyme to thus enable or facilitate operation at such temperatures. Additionally, many conventional enzymes are functional at pH of around 6.0-6.5, a pH range that is higher than the pH of the starch as conventionally provided. Typically, calcium hydroxide is used to adjust the pH and to provide the necessary calcium.

After primary liquefaction, the temperature is lowered and the enzymes are permitted to continue their activity for a longer period of time typically (90-120 minutes) at a lower temperature of around 95° C. in a secondary liquefaction step. Subsequently, a strong synthetic acid, such as sulfuric, nitric, or hydrochloric acid, is used to lower the pH to thereby deactivate the enzyme. Additional base may be added to bring the pH of the hydrolyzed starch mixture to a neutral level.

Because of the presence of strong acids and bases, substantial quantities of salts can be formed. The salts either are removed via an ion-exchange or other refining process, or are left in the starch hydrolyzate product and counted as a mineral or ash component. Ash usually is considered to be an undesired by-product, and the formation of salts thus can affect product quality or cost. Additionally, the use of strong acids and bases results in processing hazards during the starch hydrolysis reaction. Strong acids and bases also can promote the formation of reaction byproducts. It would be desirable to minimize ash formula and to minimize the use of strong acids or bases.

### SUMMARY

It has now been found that starch may be hydrolyzed at a pH ranging from about 5.0 to about 5.5 in the presence of an enzyme catalyst and calcium salt. A calcium salt may be added, but natural sources of water, even municipal clean water after the regular water treatment and purification processes, may contain 20-80 ppm of calcium, which may be sufficient for hydrolysis particularly with more thermo-stable enzymes.

The calcium salt should be a salt that provides an amount of calcium effective to fortify the enzyme catalyst without raising the pH of the starch solution more than 0.5. In many embodiments, aqueous solutions of the salt have a neutral pH. Calcium sulfate, or gypsum, is a preferred salt. In some embodiments, the enzyme may be quenched using a mild acid, one having a pK_{A} in the range of from about 2 to about 5. In other embodiments, heat is additionally or alternatively employed to quench the enzyme. The calcium salt may be, for instance, calcium sulfate, calcium phosphate, or calcium chloride, and the acid may be, for instance, citric, malic, ascorbic, acetic, or phosphoric acid. Naturally existing salts and acids are label-friendly and are preferred.

In some embodiments, the reaction will lead to less ash and better processing conditions than are obtainable using conventional enzymatic starch hydrolysis. For instance, the calcium salt in some embodiments may be used in an amount ranging from about 0.004% to about 0.5% by weight of the starch, or in some embodiments from 0.01 to about 0.1% by weight of the starch, which is several times less than the amount of calcium that is conventionally employed, and in some cases even less. The heretofore described calcium salts and acids are easier to process than are the stronger acids and bases conventionally employed. Also the materials can be “label-friendly,” thereby leading possibly to certain marketing advantages.

### BRIEF DESCRIPTION OF THE FIGURES

FIG. 1 is a schematic representation of an exemplary starch hydrolysis process.

FIG. 2 is a graph indicating the dextrose equivalent value of certain starch hydrolyzates as prepared in accordance with Example 3 hereunder.

FIGS. 3-6 are charts indicating dextrose equivalent values of starch hydrolyzates prepared in accordance with Examples 4-7 respectively.

FIG. 7 is a chart indicating a carbohydrate profile of a product prepared in accordance with Reference Example 1.

FIGS. 8-12 are charts indicating the carbohydrate profiles of products prepared in accordance with Examples 11-15 respectively.

### DESCRIPTION

The invention contemplates the hydrolysis of starch. Any suitable starch may be used in connection with the invention. Exemplary starches included corn, rice, wheat, potato, and other starches. The starch may be, for instance, a high-amylose starch, waxy starch, or, in some embodiments, may itself be in partially hydrolyzed form. In some embodiments the starch may be a chemically modified or partially derivatized starch.

Any suitable enzyme that is operable at a pH in the range of 5.0 to about 5.5 may be employed in connection with the invention. Recently, alpha-amylase enzymes derived from bacterially cloned Bacillus licheniformis have been provided commercially by NOVOZYMES. Specifically, the enzymes TERMAMYL 120L and TERMAMYL 2X have been prepared via the fermentation of the bacteria and separation of the enzyme from the fermentation product. The enzyme generally may be a non-sequenced enzyme. In some embodiments, the enzyme has an optimum activity range at a pH above about 5.9. The TERMAMYL enzymes are believed to have an optimum pH activity range of 6.0 to 6.5. Other enzymes that may be used include VALIDASE enzymes, such as VALIDASE BAA, available from DSM Valley Research, Inc. of South Bend, Ind. The VALIDASE BAA enzymes have an optimum pH activity range of 5.7 to 6.0, and are believed to have an effective pH range of 5.0-6.5 and an optimum temperature range of 83-89° C. TERMAMYL-type enzymes are believed to be enzymatically unstable below pH 5.5 in the absence of calcium at the temperatures of liquefaction described hereunder. The TERMAMYL enzymes are believed to have a half-life of under 40 minutes in the absence of calcium at temperatures of 90° C. and pH 5.5. VALIDASE and other heat-stable enzymes alternatively may be employed.

The starch is provided in the form of a slurry that may have any suitable solids percentage. In some embodiments, the slurry has a Baume measurement (a measurement of solids) of 16-20. In some embodiments, the solids concentration is 20-50%. The enzyme may be added to the starch slurry in any suitable amount effective to provide catalysis in some embodiments. An amount of 0.01%-2% active enzyme by weight of the starch is believed to be effective in some embodiments. In many embodiments, the pH of the starch slurry will range from 5.0-5.5 as supplied. In some embodiments, the pH may range from 5.0-5.6; in others, from 5.0-5.7; in others, from 5.0-5.8, and in others, from 5.0-5.9. Surprisingly, the enzymes employed are believed to be functional in many embodiments in these pH ranges.

After an addition of the enzyme, the starch slurry is then steam-jet-cooked at a temperature at around 100 to 108° C. The starch will be hydrolyzed in the presence of a calcium salt that is added to provide an initial level of a minimum 20 ppm of total free calcium ion by weight of the starch without raising the pH more than 0.5. Any suitable calcium salt may be employed, but the preferred calcium salts include the sulfate, phosphate, and chloride salts of calcium. Mixtures of the foregoing salts may be employed. In many embodiments, stronger bases such as calcium hydroxide are not used, although such stronger bases may be used if desired.

The calcium salt may be added in any suitable amount, but in some embodiments is present in an amount ranging from about 0.004 to about 0.5%, or in some embodiments 0.004 to about 0.2% by weight of the starch (measured as Ca/starch by dry weight). The calcium ion level may change as the starch is hydrolyzed. In some embodiments, the minimum initial calcium concentration is 25 ppm; in other embodiments, at least 30 ppm; in other embodiments, at least 35 ppm; in other embodiments, at least 40 ppm; in other embodiments, at least 45 ppm; in some embodiments at least 50 ppm; in some embodiments at least 60 ppm, in some embodiments at least 65 ppm, in some embodiments at least 70 ppm, in some embodiments at least 75 ppm, and in some embodiments at least 80 ppm.

In some embodiments, hydrated salts of calcium may be employed. In one embodiment, a natural product, gypsum, which is calcium sulfate dihydrate, is used to provide free calcium. Gypsum has a higher water solubility then many other calcium salts, thus leading to relatively more free calcium per weight unit. Additionally, gypsum is roughly a neutral material, thus allowing the starch slurry to remain at a pH of about 5.0 to 5.5 through the cooking and liquefaction stages. This pH range is desirable given the overall economy of the process.

In practice, the starch may be liquefied at any suitable temperature. While in some embodiments, only a primary liquefaction step is employed, generally it is desirable in some embodiments to conduct the hydrolysis in stages, the first stage being conducted at a first temperature and a second stage being conducted at a second, lower temperature. In some embodiments, starch is liquefied at a temperature ranging from about 90-120° C.; in some embodiments 90-100° C., and in some embodiments 100-108° C. in a jet-cooking step, as is conventional. The jet-cooking step may cause the slurry to be maintained at this temperature for any suitable time, such as a time ranging from 1-10 minutes; in some embodiments, 2-5 minutes.

Subsequently, in a secondary liquefaction step, the temperature may be lowered to a second, lower temperature, for instance, in the range 75 to 95° C., and in some embodiments around 95° C. In this step, the enzymes are permitted to continue their activity for a longer period of time, typically 30-150 minutes and in some embodiments 40-120 minutes. Other suitable temperatures and reaction times may be employed. The second stage may be conducted over a longer period of time than the first stage. Calcium may be present in the same amounts as heretofore described, and in some cases the liquefied mixture from the first step is subjected to secondary liquefaction without modification.

At the conclusion of the liquefaction, in general it is desired that the enzyme be quenched with an acid, optionally in the presence of heat. In some embodiments, heat alone may be used to inactivate the enzyme. In other embodiments, use of heat may reduce the amount of acid needed. Strong synthetic acids may be employed, but in many embodiments the quenching acid is a naturally derived acid. In some embodiments, the acid has a pK_{A} ranging from about 2 to about 5. For an acid with multiple pK_{A} values, the pK_{A} is deemed to be the lowest pK_{A} value for the acid in question at 25° C. Citric and phosphoric acids are useful in some embodiments in connection with the quenching step. The acid may be used in any suitable amount, but typically is provided in an amount sufficient to lower the pH of the liquefied starch mixture to a pH in the range of about 3.8 to 4.0.

The liquefaction may be continued to any suitable extent. In some embodiments, the starch liquefaction may be carried out essentially completely to form principally free glucose. In other embodiments, a corn syrup having a dextrose equivalent (DE) value greater than 75 may be provided. In other embodiments, liquefaction may be carried out to provide a product having a dextrose equivalent value greater than 50 but less than 75. In other embodiments, the hydrolysis may be connected to an extent sufficient to provide a product having a dextrose equivalent value of 50, or of less than 50. In other embodiments, the hydrolysis may be conducted to an extent sufficient to provide a dextrose equivalent value of less than 20. In other embodiments, the hydrolysis may be conducted to an extent sufficient to provide a product having a dextrose equivalent value greater than or equal to 20. Generally, mixtures of oligosaccharides having a dextrose equivalent value of less than 20 are deemed to maltodextrins, while those having a dextrose equivalent value greater than or equal to 20 are deemed to be syrup solids. In some embodiments, the hydrolysis may be conducted to an extent sufficient to provide a product having a dextrose equivalent value in the range of 4 to 18. The measurement of dextrose equivalent value may be conducted in any suitable manner, such as the Schoorl method or the osmolality method.

In some embodiments, maltodextrins having a DP 1-8 profile similar to those in the following table may be prepared (DP signifying the degree of polymerization).

Typical DP Profile (% dry solids basis)

| DP Profile | M180 | M150 | M100 | M050 | M040 |
|---|---|---|---|---|---|
| DP >8 | 46.6 ± 4% | 54.7 ± 4% | 67.8 ± 4% | 90.6 ± 4% | 88.5 ± 4% |
| DP 8 | 3.9 ± 2% | 4.8 ± 1.5% | 4.5 ± 1.5% | 1.5 ± 1% | 2.0 ± 1% |
| DP 7 | 9.5 ± 2% | 9.1 ± 1.5% | 7.0 ± 1.5% | 1.5 ± 1% | 2.4 ± 1% |
| DP 6 | 11.4 ± 2% | 8.4 ± 1.5% | 6.1 ± 1.5% | 1.4 ± 1% | 1.8 ± 1% |
| DP 5 | 5.9 ± 2% | 4.7 ± 1.5% | 3.3 ± 1.5% | 1.3 ± 1% | 1.3 ± 1% |
| DP 4 | 6.4 ± 2% | 5.5 ± 1.5% | 3.7 ± 1.5% | 1.1 ± 1% | 1.4 ± 1% |
| DP 3 | 8.3 ± 2% | 6.7 ± 1.5% | 4.2 ± 1.5% | 1.0 ± 1% | 1.4 ± 1% |
| DP 2 | 6.2 ± 2% | 4.8 ± 1% | 2.5 ± 1% | 0.8* ± 1% | 0.9* ± 1% |
| DP 1 | 1.8 ± 1.5% | 1.3 ± 1% | 0.7* ± 1% | 0.8* ± 1% | 0.3* ± 1% |

*MINIMUM VALUE = 0%

After formation of the starch hydrolyzate product, the product may be decolored, such as with activated carbon, and solids removed via filtration. The resulting solution may be filtered and dried to a moisture content of 10% or less to form a dry product.

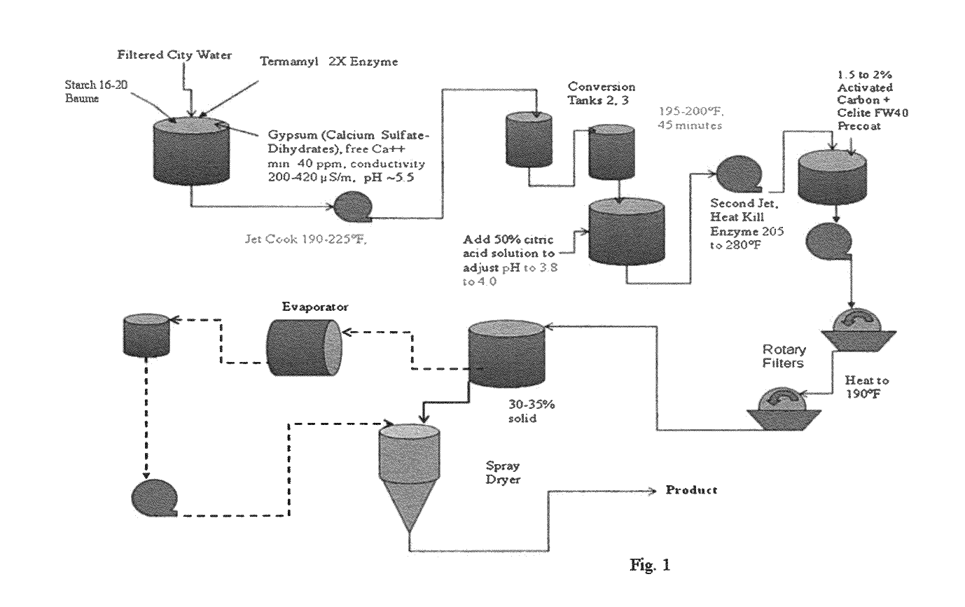

With respect to the exemplary embodiment illustrated in FIG. 1, it is seen that filtered city water (Muscatine, Iowa) and starch are added with gypsum and an enzyme to a stirring tank. The mixture is jet-cooked and held in conversion tanks for secondary liquefaction. Citric acid is added in a stirring tank, and secondary heat is applied to kill the enzyme. Activated carbon is added, and the mixture is filtered in a precoat filter. Subsequently, the mixture is further filtered in first and second rotary filters. The final starch hydrolyzate product then optionally may be evaporated, or may be introduced as-is to a spray drier.

The following examples are provided. These examples should not be deemed as limiting the invention in scope.

### Example 1

Starch milk from a corn wet milling process was diluted to 20 Baume in concentration (35.54% dry solid starch). A shiny of gypsum was added to bring the calcium content to between 70 to 80 ppm, or conductivity to 200-220 microS. Then, TERMAMYL 2X enzyme (as-is solution) was added to the starch slurry in an amount of 0.04% wt. based on total starch weight.

The starch slurry was then cooked using a steam jet cooker at a temperature of around 220° F. for 5 minutes. The cooked starch was held in containers for 40 to 45 minutes at 195 to 200° F. to allow the enzyme to hydrolyze the starch. Samples were taken to measure DE of the starch hydrolyzates. When a target DE was achieved, a solution of citric acid (50% NON) solution was added to the slurry to bring its pH to 3.8 to 4.0. Then the starch hydrolyzate slurry was cooked in a jet cooker at 210° F. to 220° F. to inactivate the enzyme. Activated carbon, SA-1500 from MeadWestvaco Corporation, in an amount of 1.5% by dry solids weight of the starch hydrolyzate, was then mixed into the enzyme-inactivated starch hydrolyzate slurry. The slurry was held at 185° F. for 30 min with mixing. Then the slurry was filtered using a rotary filter with CELATOM Diatomaceous Earth FW 40 pre-coat filter aid to remove insoluble materials. The filtrate was then collected and spray-dried. The products produced had a DE ranging from 3 to 25 depending on time of the secondary liquefaction.

### Example 2

Starch milk from a corn wet milling process was diluted to 17.5 Baume in concentration (31.098% dry solid starch). A slurry of gypsum was added to bring the calcium content to 70 to 80 ppm, or conductivity to 200-220 microS. Then, TERMAMYL 2X enzyme (as-is solution) was added to the starch slurry at an amount of 0.12% wt. based on total starch weight. The starch slurry was then cooked using a steam jet cooker with temperature setting at around 215° F. for 5 minutes. The jet cooker had a capacity of 2.5 gallon per min.

The cooked starch was held in containers for 40 to 45 minutes around 205° F. to allow the enzyme to hydrolyze the starch. Samples were taken to measure DE of the starch hydrolyzates. When a target DE was achieved, a solution of citric acid (50% w/w) solution was to the slurry to bring its pH to 3.8 to 4.0. Then the starch hydrolyzate slurry was cooked in a jet cooker at 205° F. to inactivate the enzyme. SA-1500 activated carbon was added in an amount of 1.5% by dry solids weight of the starch hydrolyzate. The slurry was held at 195° F. for 30 min with mixing. Then the slurry was filtered using the rotary filters with CELITE Diatomaceous Earth FW 40 pre-coat filter aid to remove insoluble materials. The filtrate was then collected and reheated using a heat-exchanger to 190° F., then filtered through second rotary filters with CELITE Diatomaceous Earth FW 40 pre-coat filter aid to further remove insoluble material. The filtrate collected was then further refined with a set of CUNO filter with 10 micron cartridges. The filtrates were either concentrated to around 50% solid using an evaporator, then spray-dried, or spray-dried without a prior concentration process. The products produced had a DE range from 3 to 25.

### Example 3

Starch milk from a corn wet milling process was diluted to 20 Baume in concentration (35.540% dry solid starch). The starch milk had a pH of 5.35 and conductivity of 138 microS. A saturated slurry of gypsum (20 grams in 500 ml water) was added to adjust the conductivity to 234 microS and a calcium content of 65 ppm. Then, TERMAMYL 2X enzyme (as-is solution) was added to the starch slurry in an amount of 0.04% wt. based on total starch weight. The starch slurry was then cooked using a small bench-top steam jet cooker with temperature setting at around 220-221(+/−) 5° F. for 40 seconds. The jet cooker had a capacity of 0.185 gallon per min.

The cooked starch was held in containers at around 195° F. in water bath to allow the enzyme to hydrolyze the starch, Samples were taken to measure DE of the starch hydrolyzates after 30 min, 70 min, 90 ml, 110 min, and 150 min of conversion time respectively. Then the starch hydrolyzate slurry was cooked in a jet cooker at 270° F. to inactivate the enzyme.

SA-1500 activated carbon was added at 1.5% by weight of starch hydrolyzate solid weight by mixing. The slurry was held at 185° F. for 30 min with mixing. Then the slurry was filtered using a Buchner funnel filters with CELATOM Diatomaceous Earth FW 40 pre-coat filter aid to remove insoluble material. The filtrate was then collected and filtered again through a No. 1 Whatman filter paper. The filtrate was spray-dried using a Bowen Dryer, with setting at inlet temperature: 360° F., discharge temperature 190° F. The products produced had a DE range from 3 to 25 depended on the conversion time.

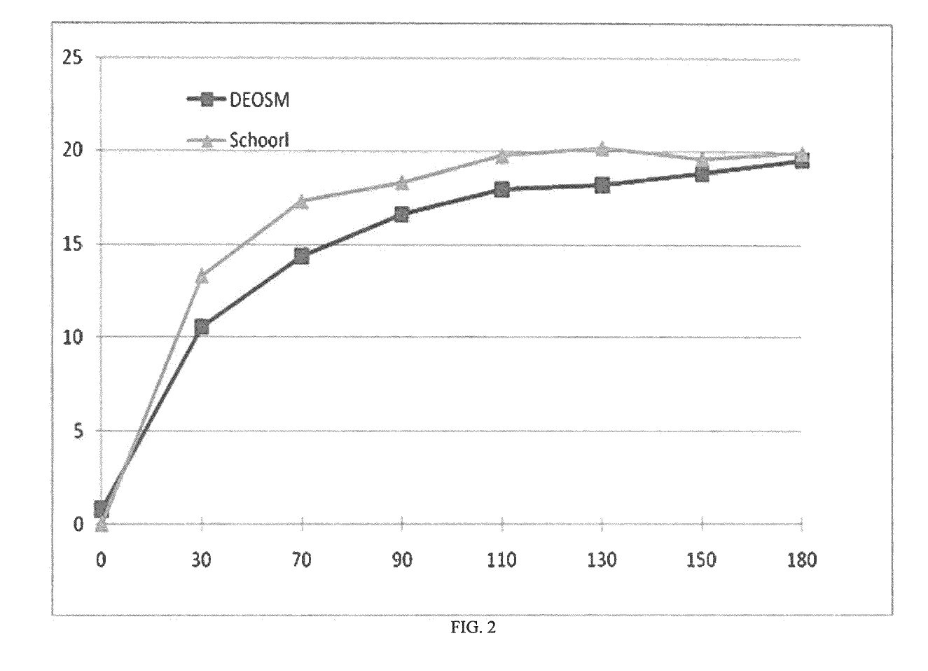

FIG. 2 illustrates the DE of the products as a function of time. As indicated, the DE was measured in accordance with two techniques.

### Example 4

Starch milk from a corn wet milling process was diluted to 20.2 Baume in concentration (35.895% dry solid starch). The starch milk had a pH of 5.5 and a conductivity of 135 microS. A saturated slurry of gypsum (20 grams in 500 ml water) was added to adjust conductivity to 237 microS and a calcium content of 85 ppm. Then, TERMAMYL 2X enzyme (as-is solution) was added to the starch slurry at a rate 0.05% wt. based on total starch weight. The starch slurry was then cooked using a small bench-top steam jet cooker with temperature setting at around 220-221(+/−) 5° F. for 5 minutes. The jet cooker had a capacity of 0.185 gallon per min.

The cooked starch was held in a set of four steam-jacketed boxes at around 195° F. to allow the enzyme to hydrolyze the starch. After a total of 40 minutes (as measured from the first jet cooking), a solution of citric acid (50% w/w) was added to the slurry to bring its pH to 3.8 to 4.0. Then the starch hydrolyzate slurry was cooked in a jet cooker at 210° F. to inactivate the enzyme.

SA-1500 activated carbon was added at 1.5% by weight of starch hydrolyzate solid weight by mixing. The slurry was held at 185° F. for 30 min with mixing. Then the slurry was filtered using a Buchner funnel filters with CELATOM Diatomaceous Earth FW 40 pre-coat filter aid to remove insoluble material. The filtrate was then collected and filtered again through a No. 1 Whatman filter paper. The filtrate was spray-dried using a Bowen Dryer, with the following settings at inlet temperature: 360° F., discharge temperature 190° F. The product produced had a DE range 14.9 (Schoorl method).

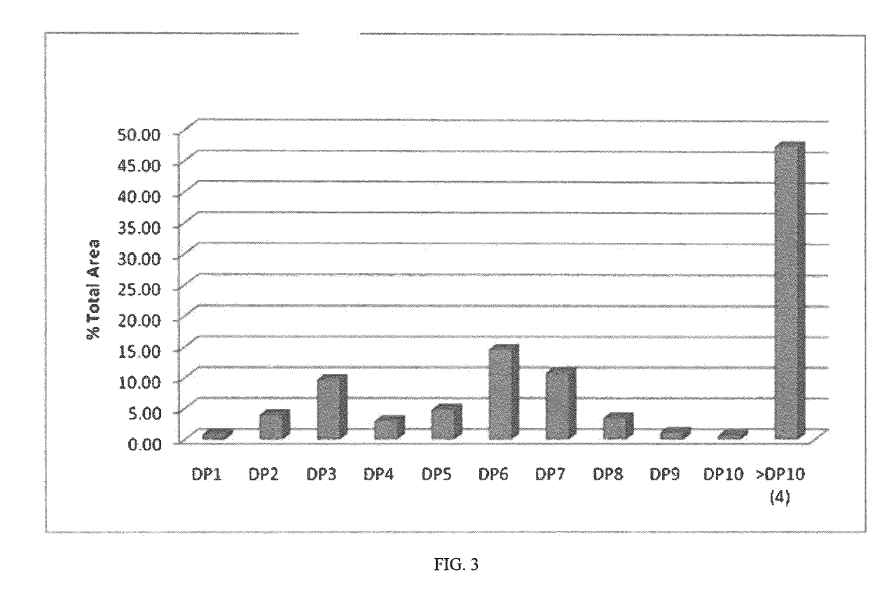

The product had the carbohydrate profile shown in FIG. 3.

### Example 5

Starch milk from a corn wet milling process was diluted to 20.2 Baume in concentration (35.895% dry solid starch). The starch milk had a pH of 5.5 and a conductivity of 135 microS. A saturated slurry of gypsum (20 grams in 500 ml water) was added to adjust the conductivity to 237 microS and the calcium content to 85 ppm. Then, TERMAMYL 2X enzyme (as-is solution) was added to the starch slurry in the amount of 0.06% wt. based on total starch weight. The starch slurry was then cooked using a small bench-top steam jet cooker at a temperature of around 220-221(+/−) 5° F. for 5 minutes. The jet cooker had a capacity of 0.185 gallon per min.

The cooked starch was held in a set of four steam-jacketed boxes at around 195° F. to allow the enzyme to hydrolyze the starch. After 40 minutes, a solution of citric acid (50% w/w) was added to the slurry to bring its pH to 3.8 to 4.0. Then the starch hydrolyzate slurry was cooked in a jet cooker at 210° F. to inactivate the enzyme.

Activated carbon (SA-1500) was added at 1.5% by weight of starch hydrolyzate solid weight by mixing. The slurry was held at 185° F. for 30 min with mixing. Then the slurry was filtered using a Buchner funnel filters with CELATOM Diatomaceous Earth FW 40 pre-coat filter aid to remove insoluble material. The filtrate was then collected and filtered again through a No. 1 Whatman filter paper. The filtrate was spray-dried using a Bowen Dryer, with the following settings at inlet temperature: 360° F., discharge temperature 190° F. The products produced had a DE of 17.0 as measured by the Schoorl method.

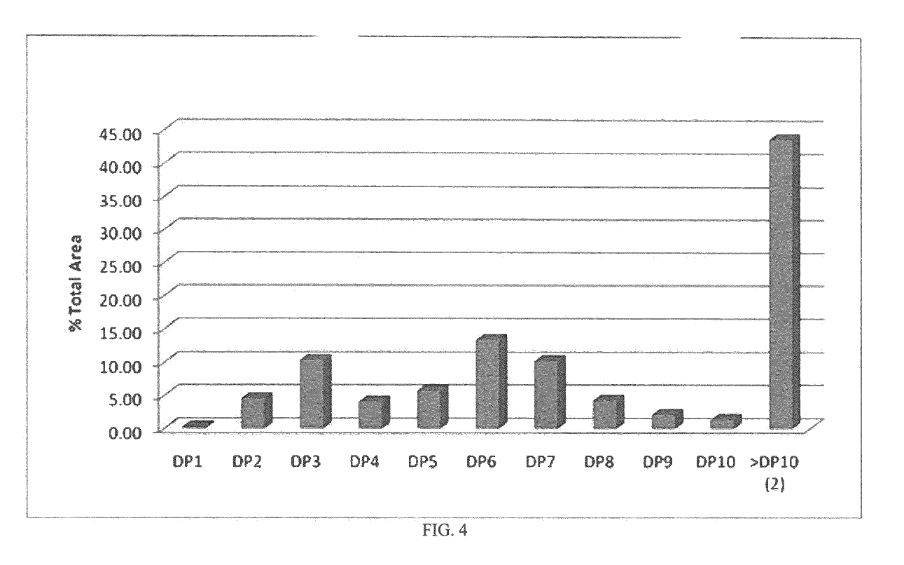

The product had the carbohydrate profile shown in FIG. 4.

### Example 6

Starch milk from a corn wet milling process was diluted to 20 Baume in concentration (35.54% dry solid starch). The starch milk had a pH of 5.6 and a conductivity of 135 microS. A saturated slurry of gypsum (20 grams in 500 ml water) was added to bring the conductivity to 223 microS and the calcium content to 89 ppm. Then, TERMAMYL 2X enzyme (as-is solution) was added to the starch slurry in an amount of 0.02% wt based on total starch weight. The starch slurry was then cooked using a small bench-top steam jet cooker at a temperature of around 220-221(+/−) 5° F. for 5 minutes. The jet cooker had a capacity of 0.185 gallon per min.

The cooked starch was held in a set of four steam-jacketed boxes at around 195° F. to allow the enzyme to hydrolyze the starch. After 40 minutes, a solution of citric acid (50% w/w) was added to the slurry to bring its pH to 3.8 to 4.0. Then the slurry was cooked in a jet cooker at 210° F. to inactivate the enzyme.

Activated carbon (SA-1500) was added at 1.5% by weight of starch hydrolyzate solid weight by mixing. The slurry was held at 185° F. for 30 min with mixing. Then the slurry was filtered using a Buchner funnel filters with CELATOM Diatomaceous Earth FW 40 pre-coat filter aid to remove insoluble material. The filtrate was then collected and filtered again through a No. 1 Whatman filter paper. The filtrate was spray-dried using a Bowen Dryer, with setting at inlet temperatures: 360° F., discharge temperature 190° F. The products produced had a DE of 16.8 as measured via the Schoorl method.

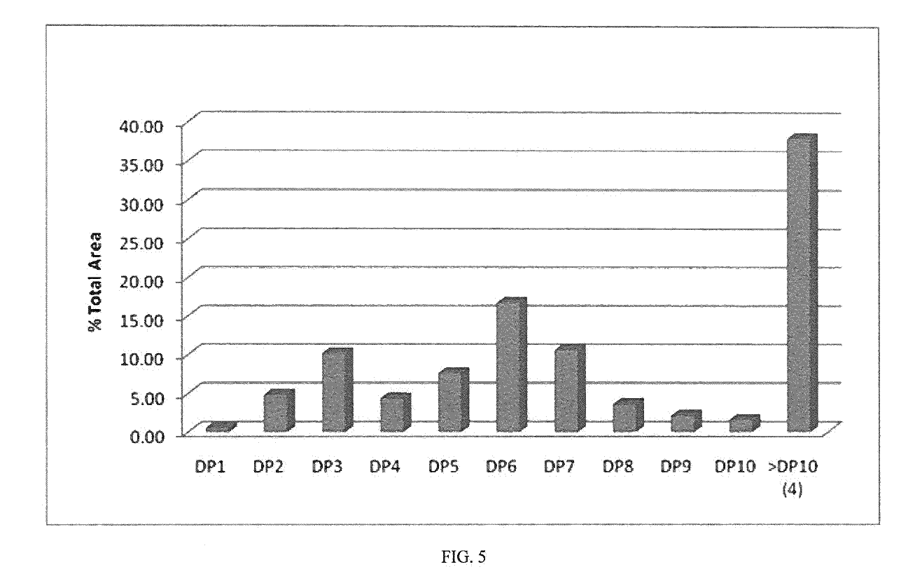

The product had the carbohydrate shown in FIG. 5.

### Example 7

Starch milk from a corn wet milling process was diluted to 20 Baume in concentration (35.54% dry solid starch). The starch milk had a pH of 5.6. A saturated slurry of gypsum (20 grams in 500 nil water) was added to adjust the conductivity to 244 microS and a calcium content of 89 ppm. Then, TERMAMYL 2X enzyme (as-is solution) was added to the starch slurry in an amount of 0.58 grams per gallon of slurry. The starch slurry was then cooked using a small bench-top steam jet cooker at a temperature of around 220-221(+/−)5° F. for 5 minutes. The jet cooker had a capacity of 0.185 gallon per min.

The cooked starch was held in a set of four steam-jacketed boxes at around 195° F. to allow the enzyme to hydrolyze the starch. After 40 minutes from the first jet cooking, a solution of citric acid (50% w/w) was added to the slurry to bring its pH to 3.8 to 4.0. Then the starch hydrolyzate slurry was cooked in a jet cooker at 210° F. to inactivate the enzyme.

Activated carbon (SA-1500) was added at 1.5% by weight of starch hydrolyzate solid weight by mixing. The slurry was held at 185° F. for 30 min with mixing. Then the slurry was filtered using a Buchner funnel filters with CELATOM Diatomaceous Earth FW 40 pre-coat filter aid to remove insoluble material. The filtrate was then collected and filtered again through a No. 1 Whatman filter paper. The filtrate was spray-dried using a Bowen Dryer, with setting at inlet temperatures: 360° F., discharge temperature 190° F. The products produced had a DE of 15 by the Schoorl method.

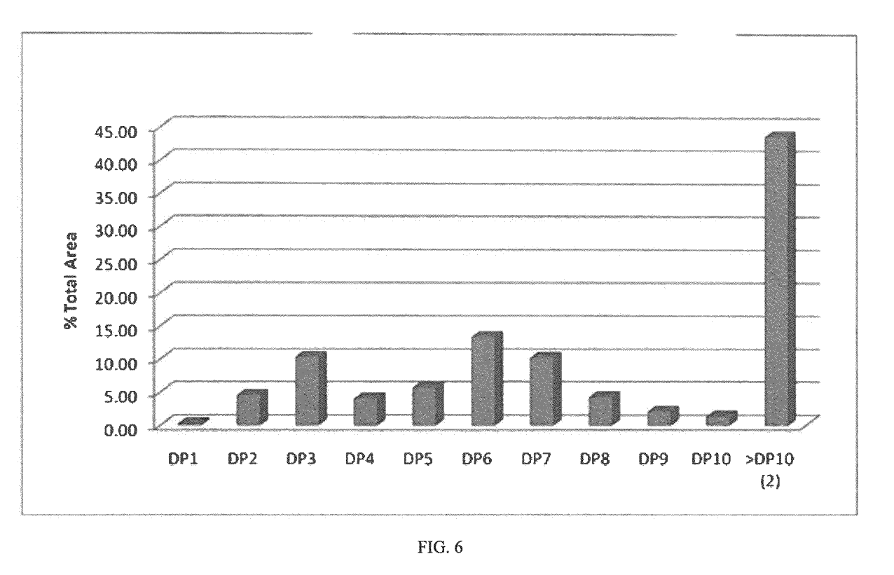

The product had the carbohydrate profile illustrated in FIG. 6.

### Example 8

Starch milk from a corn wet milling process was diluted to 14.5 Baume in concentration (25.767% dry solid starch). The starch milk had a pH of 5.5. A slurry of gypsum was added to adjust the conductivity to 400 microS and a calcium content higher than 120 ppm. Then, TERMAMYL 2X enzyme (as-is solution) was added to the starch slurry at rate between 0.09% and 0.12% of starch dry weight. The starch slurry was then cooked using a hydroheater with temperature setting at around 210-216° F. for 30 minutes. The hydroheater was running at around 88 to 90 gallon of starch milk per min.

The cooked starch was held in steam-jacketed conversion tanks at a temperature of around 205° F. to allow the enzyme to hydrolyze the starch. TERMAMYL 2X enzyme in an amount of 0.0125% by starch weight was added into the first conversion tank in some cases. After 120 minutes, a solution of citric acid (50% w/w) was added to the slurry to bring its pH to 3.8 to 4.0. Then the starch hydrolyzate slurry was cooked in a jet cooker at 205° F. to inactivate the enzyme.

Activated carbon SA-1500 from MeadWestvaco Corporation, at 1.5% weight of starch hydrolyzate solid weight, was then mixed into the enzyme-inactivated starch hydrolyzate slurry. The slurry mixture was held at 185° F. or above for about 30 min with mixing. The slurry then was filtered using rotary filters with CELITE Diatomaceous Earth FW 40 pre-coat filter aid to remove insoluble material, and then filtered through second rotary filters with CELITE Diatomaceous Earth FW 40 pre-coat filter aid to further refine the filtrates. The filtrate was then collected and reheated using a heat-exchanger to above 180° F., then further refined with a set of Niagara filters with 10 micron cartridges. The filtrates were either concentrated to around 50% solid using an evaporator, then spray-dried, or spray-dried without concentration process. The products produced had a DE range from 10 to 18 depending on enzyme dosages and conversion time after the first hydroheater cooking.

### Reference Example 1

This Example illustrates that TERMAMYL enzymes are active at a pH of about 6.4.

Tapioca starch was diluted with city water to 12 Baume in concentration (21.324% dry solid starch). The starch milk had a pH of 6.4 and conductivity of 334 microS. A saturated slurry of gypsum (20 grams in 500 ml water) was added to adjust the conductivity to 400 microS. Then, TERMAMYL 2X enzyme (as-is solution) was added to the starch slurry in an amount of 0.048% wt. based on total starch weight. The starch slurry was then cooked using a small bench-top steam jet cooker with temperature setting at around 220-221(+/−) 5° F. for 5 minutes. The jet cooker had a capacity of 0.185 gallon per min.

The cooked starch was held in a set of two steam-jacketed boxes at around 195° F. to allow the enzyme to hydrolyze the starch. After the slurry flow from the first box in about 12 minutes, a solution of citric acid (50% w/w) was added to the slurry in the second box to bring its pH to 3.8 to 4.0. Then the starch hydrolyzate slurry was cooked in a jet cooker at 210° F. to inactivate the enzyme.

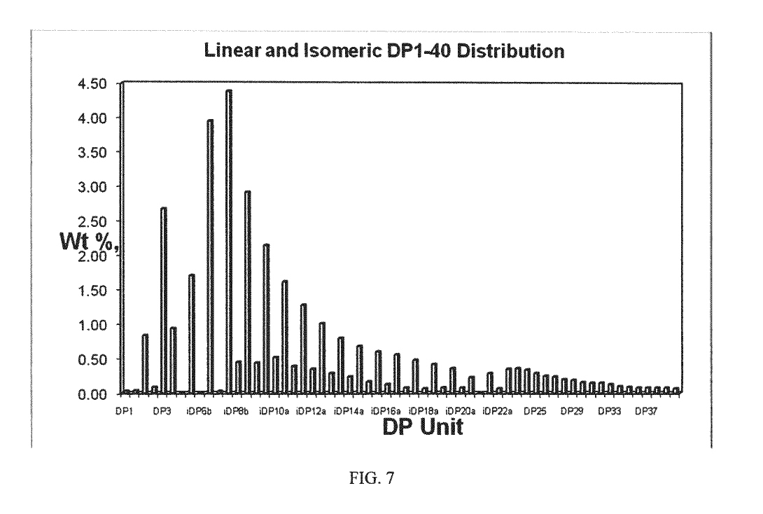

Activated carbon (SA-1500) was added at 1.5% by weight of starch hydrolyzate solid weight by mixing. The slurry was held at 185° F. for 30 min with mixing. Then the slurry was filtered using a Buchner funnel filters with CELATOM Diatomaceous Earth FW 40 pre-coat filter aid to remove insoluble material. The filtrate was then collected and filtered again through a No. 1 Whatman filter paper. The filtrate was spray-dried using a Bowen Dryer, with settings at inlet temperature: 360° F., discharge temperature 190° F. The product had the carbohydrate profile shown in FIG. 7. DE of product was estimated at around 4.5 to 5.0 by a calculation based on carbohydrate profile.

### Example 9

Starch milk from a corn wet milling process is diluted to 14.5 Baume in concentration (25.767% dry solid starch). The starch milk has a pH of below than 5.0. Trona (sodium sesquicarbonate, Na3H(CO3)2.2H2O), or soda ash is added to adjust pH to 5.5 to 6.5. A slurry of gypsum is added to adjust conductivity to 400 microS and a calcium content of higher than 120 ppm. TERMAMYL 2X enzyme (as-is solution) is added to the starch slurry at rate between 0.02% and 0.12% of starch dry weight. The starch slurry is then cooked using hydroheater at a temperature of around 210-216° F. for 30 minutes. The hydroheater runs at around 88 to 90 gallon of starch milk per min.

The cooked starch is held in steam-jacketed conversion tanks at around 205° F. to allow the enzyme to hydrolyze the starch. A dose of TERMAMYL 2X enzyme, in an amount of 0.0125% by starch weight, is added into the first conversion tank in some cases. After 120 minutes, a solution of citric acid (50% w/w) is added to the slurry to bring its pH to 3.8 to 4.0. The slurry then is cooked in a jet cooker at 205° F. to inactivate the enzyme.

Activated carbon SA-1500 from MeadWestvaco Corporation, at 1.5% weight of starch hydrolyzate solid weight, is then mixed into the enzyme-inactivated starch hydrolyzate slurry. The slurry mixture is held at 185° F. or above for about 30 min with mixing.

Then the slurry is filtered using first rotary filters with CELITE Diatomaceous Earth FW 40 pre-coat filter aid to remove insoluble material, and then filtered through second rotary filters with CELITE Diatomaceous Earth FW 40 pre-coat filter aid to further refine the filtrates. The filtrate is then collected and reheated using a heat-exchanger to above 180° F., then further refined with a set of Niagara filters with 10 micron cartridges. The filtrates are either concentrated to around 50% solid using an evaporator, then spray-dried, or spray-dried without prior concentration process. The products produced have a DE ranging from 10 to 18 depending on enzyme dosages and conversion time after the first hydroheater cooking.

### Example 10

In processes described in Examples 1 to 9, ascorbic acid or malic acid, or acetic acid, or vinegar or other organic acids, instead of citric acid, is used to adjust the pH to 3.8 to 4.0 before the second jet cooking to inactivate the enzyme.

### Example 11

Starch milk from a corn wet milling process was diluted to 15.2 Baume in concentration. The city water contains 63.19 ppm (as is) calcium. The diluted starch milk has a calcium content of 28.47 ppm and pH 5.91. Then, VALIDASE BAA enzyme was added to the starch slurry in an amount of 0.023% of based on corn starch weight. The starch slurry was then cooked using pilot size steam jet cooker at a temperature of around 225-227±2° F. for 10 minutes. The starch was fed at 3 gal/min or 180 gal/hr to the cooker.

The cooked starch was held in a steam-jacketed conversion tank with temperature maintained around 203-205° F. to allow the enzyme to hydrolyze the starch. After 90 to 100 minutes from the first jet cooking, a solution of citric acid (5% w/w) was added to the slurry to bring its pH to 4.0 to 4.2. Then the starch hydrolyzate slurry was cooked in a jet cooker at 215 to 220° F. to inactivate the enzyme.

The converted slurry was collected and held in a tank, and then pre-mixed slurry of SA-1500 Activated Carbon and CELATOM Diatomaceous Earth FW 14 pre-coat filter aid was added into the tank. Activated carbon is used at the ratio of 1.0 to 1.5% of solids in the starch slurry. The mixture was then filtered through a Rotary Vacuum Filter equipment pre-coated with CELATOM Diatomaceous Earth FW 140 filter aid. The filtrate was further then further refined with a set of CUNO filter with 10 micron cartridges. The filtrate was then collected then concentrated with around 50% solids using an evaporator. The concentrated filtrate was then spray-dried using a Spray Dryer, with setting of 380-400° F. inlet temperature (T1) and 200° F. outlet temperature. The products produced had a DE of 14.05 by the Schoorl method.

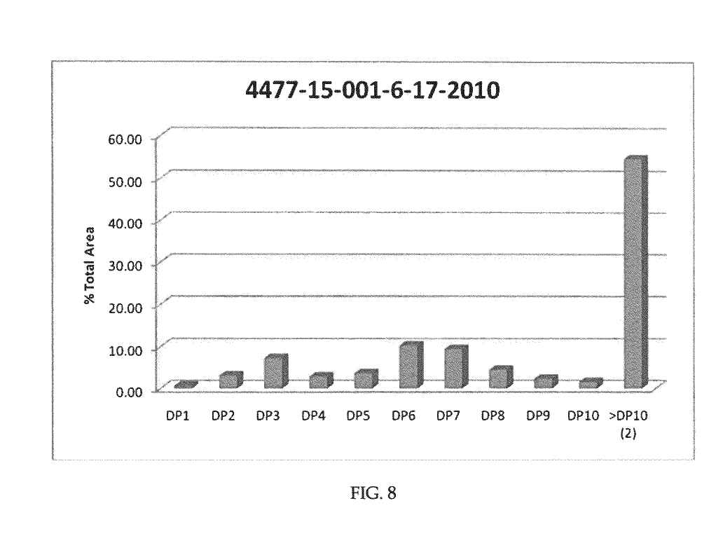

The product had the carbohydrate profile illustrated in FIG. 8.

### Example 12

Starch milk from a corn wet milling process was diluted to 15.8 Baume in concentration. The city water contains 55.79 ppm (as is) calcium. The diluted starch milk has a calcium content of 22.72 ppm and pH 6.08. Then, VALIDASE BAA enzyme was added to the starch slurry in an amount of 0.039% of based on corn starch weight. The starch slurry was then cooked using pilot size steam jet cooker at a temperature of around 225-227±2° F. for 10 minutes. The starch was fed at 3 gal/min or 180 gal/hr to the cooker.

The cooked starch was held in a steam-jacketed conversion tank with temperature maintained around 175° F. to allow the enzyme to hydrolyze the starch. After 90 to 100 minutes from the first jet cooking, a solution of citric acid (5% w/w) was added to the slurry to bring its pH to 4.0 to 4.2. Then the starch hydrolyzate slurry was cooked in a jet cooker at 215 to 220° F. to inactivate the enzyme.

The converted slurry was collected and held in a tank, and then pre-mixed slurry of SA-1500 Activated Carbon and CELATOM Diatomaceous Earth FW 14 pre-coat filter aid was added into the tank. Activated carbon was used at the ratio of 1.0 to 1.5% of solids in the starch slurry. The mixture was then filtered through a Rotary Vacuum Filter equipment pre-coated with CELATOM Diatomaceous Earth FW 140 filter aid. The filtrate was further then further refined with a set of CUNO filter with 10 micron cartridges. The filtrate was then collected then concentrated with around 50% solids using an evaporator. The concentrated filtrate was then spray-dried using a Spray Dryer, with setting of 380-400° F. inlet temperature (T1) and 200° F. outlet temperature. The products produced had a DE of 17.8 by the Schoorl method.

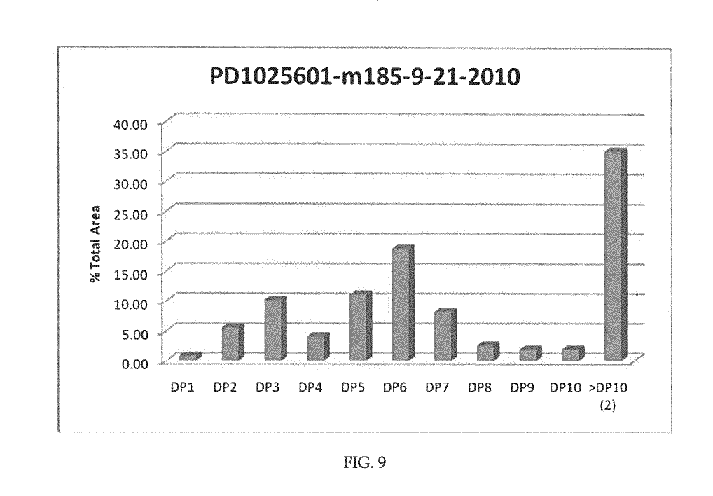

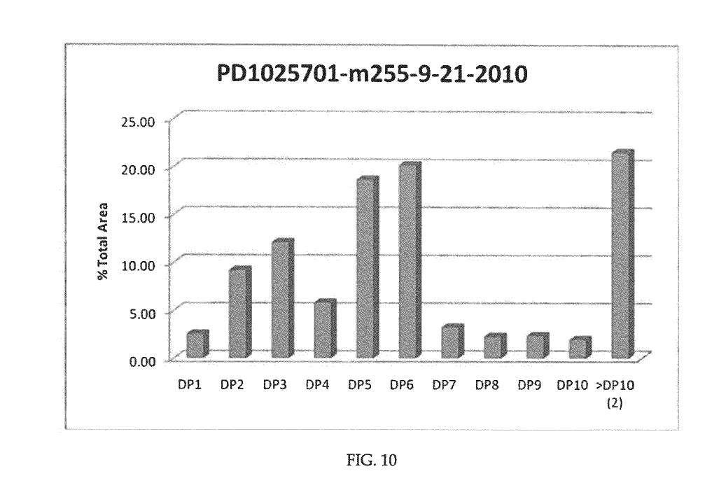

The product had the carbohydrate profile illustrated in FIG. 9.

### Example 13

Starch milk from a corn wet milling process was diluted to 16 Baume in concentration. The city water contains 55.79 ppm (as is) calcium. The diluted starch milk has a calcium content of 22.72 ppm and pH 6.1. Then, VALIDASE BAA enzyme was added to the starch slurry in an amount of 0.083% of based on corn starch weight. The starch slurry was then cooked using pilot size steam jet cooker at a temperature of around 225-227±2° F. for 10 minutes. The starch was fed at 3 gal/min or 180 gal/hr to the cooker.

The cooked starch was held in a steam-jacketed conversion tank with temperature maintained around 190-195° F. to allow the enzyme to hydrolyze the starch. After 90-100 minutes from the first jet cooking, a solution of citric acid (5% w/w) was added to the slurry to bring its pH to 4.0 to 4.2. Then the starch hydrolyzate slurry was cooked in a jet cooker at 215 to 220° F. to inactivate the enzyme.

The converted slurry was collected and held in a tank, and then pre-mixed slurry of SA-1500 Activated Carbon and CELATOM Diatomaceous Earth FW 14 pre-coat filter aid was added into the tank. Activated carbon was used at the ratio of 1.0 to 1.5% of solids in the starch slurry. The mixture was then filtered through a Rotary Vacuum Filter equipment pre-coated with CELATOM Diatomaceous Earth FW 140 filter aid. The filtrate was further then further refined with a set of CUNO filter with 10 micron cartridges. The filtrate was then collected then concentrated with around 50% solids using an evaporator. The concentrated filtrate was then spray-dried using a Spray Dryer, with setting of 380-400° F. inlet temperature (T1) and 200° F. outlet temperature. The products produced had a DE of 24.1 by the Schoorl method.

The product had the carbohydrate profile illustrated in FIG. 9.

### Example 14

Starch milk from a corn wet milling process was diluted to 15.2 Baume in concentration. The city water contains 55.79 ppm (as is) calcium. The diluted starch milk has a calcium content of 22.72 ppm and pH 6.1. Then, VALIDASE BAA enzyme was added to the starch slurry in an amount of 0.0090% of based on corn starch weight. The starch slurry was then cooked using pilot size steam jet cooker at a temperature of around 225-227±2° F. for 10 minutes. The starch was fed at 3 gal/min or 180 gal/hr to the cooker.

The cooked starch was held in a steam-jacketed conversion tank with temperature maintained around 190-200° F. to allow the enzyme to hydrolyze the starch. After 120 minutes from the first jet cooking, a solution of citric acid (5% w/w) was added to the slurry to bring its pH to 4.0 to 4.2. Then the starch hydrolyzate slurry was cooked in a jet cooker at 215 to 220° F. to inactivate the enzyme.

The converted slurry was collected and held in a tank, and then pre-mixed slurry of SA-1500 Activated Carbon and CELATOM Diatomaceous Earth FW 14 pre-coat filter aid was added into the tank. Activated carbon was used at the ratio of 1.0 to 1.5% of solids in the starch slurry. The mixture was then filtered through a Rotary Vacuum Filter equipment pre-coated with CELATOM Diatomaceous Earth FW 140 filter aid. The filtrate was further then further refined with a set of CUNO filter with 10 micron cartridges. The filtrate was then collected then concentrated with around 50% solids using an evaporator. The concentrated filtrate was then spray-dried using a Spray Dryer, with setting of 380-400° F. inlet temperature (T1) and 200° F. outlet temperature. The products produced had a DE of 7.5 by the Schoorl method.

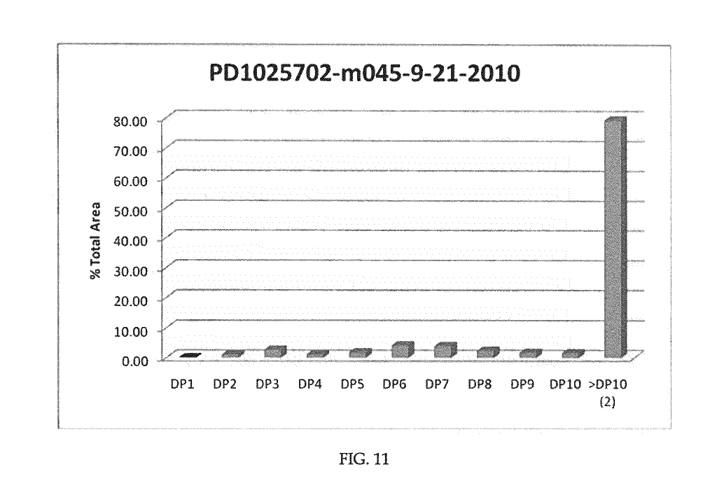

The product had the carbohydrate profile illustrated in FIG. 11.

### Example 15

Starch milk from a corn wet milling process was diluted to 16 Baume in concentration. The city water contains 54.46 ppm (as is) calcium. The diluted starch milk has a calcium content of 28.20 ppm and pH 5.8. Then, VALIDASE BAA enzyme, an alpha-amylase from Valley Research, was added to the starch slurry in an amount of 0.0099% of based on corn starch weight. The starch slurry was then cooked using pilot size steam jet cooker at a temperature of around 225-227±2° F. for 10 minutes. The starch was fed at 3 gal/min or 180 gal/hr to the cooker.

The cooked starch was held in a steam-jacketed conversion tank with temperature maintained around 190-200° F. to allow the enzyme to hydrolyze the starch. After 120 minutes from the first jet cooking, a solution of citric acid (5% w/w) was added to the slurry to bring its pH to 4.0 to 4.2. Then the starch hydrolyzate slurry was cooked in a jet cooker at 215 to 220° F. to inactivate the enzyme.

The converted slurry was collected and held in a tank, and then pre-mixed slurry of SA-1500 Activated Carbon and CELATOM Diatomaceous Earth FW 14 pre-coat filter aid was added into the tank. Activated carbon was used at the ratio of 1.0 to 1.5% of solids in the starch slurry. The mixture was then filtered through a Rotary Vacuum Filter equipment pre-coated with CELATOM Diatomaceous Earth FW 140 filter aid. The filtrate was further then further refined with a set of CUNO filter with 10 micron cartridges. The filtrate was then collected then concentrated with around 50% solids using an evaporator. The concentrated filtrate was then spray-dried using a Spray Dryer, with setting of 380-400° F. inlet temperature (T1) and 200° F. outlet temperature. The products produced had a DE of 7.38 by the Schoorl method.

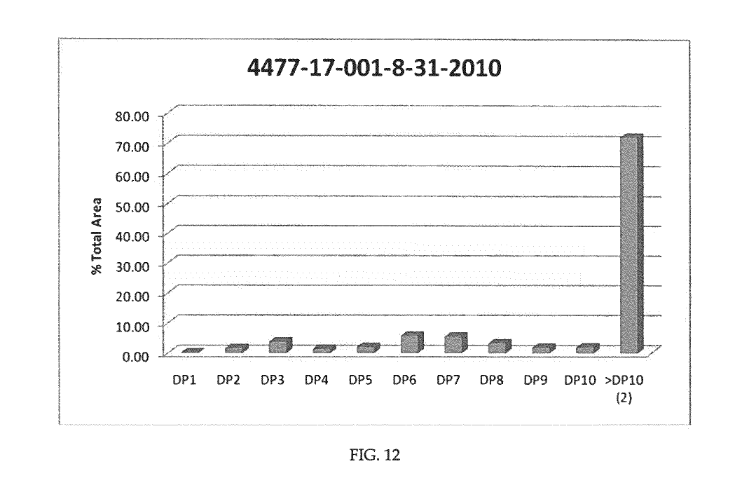

The product had the carbohydrate profile illustrated in FIG. 12.

It is thus seen that a starch hydrolysis method may be performed and can achieve certain advantages over the prior methods herein described.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain embodiments as “preferred” embodiments, and other recitation of embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

1. A method for hydrolyzing starch comprising:
providing a starch:
hydrolyzing said starch at a pH ranging from about 5.0 to about 5.5 in the presence of an enzyme catalyst and a neutral calcium salt to yield a hydrolyzed starch product.

2. A method according to claim 1, said calcium salt being selected from the group consisting of the sulfate, phosphate, and chloride salts of calcium, and mixtures thereof.

3. A method according to claim 1, said calcium salt being present in an amount ranging from about 0.004 to 0.5% by weight of the starch.

4. A method according to claim 1, further comprising deactivating said enzyme with an acid having a pK_{A} ranging from about 2 to about 5.

5. A method according to claim 1, further comprising deactivating said enzyme with heat.

6. A method according to claim 4, said acid being selected from the group consisting of citric, acetic, ascorbic, malic, and phosphoric acids.

7. A method according to claim 1, said hydrolysis being conducted to an extent sufficient to provide a product having a dextrose equivalent value greater than 75.

8. A method according to claim 1, said hydrolysis being conducted to an extent sufficient to provide a product having a dextrose equivalent value greater than 50.

9. A method according to claim 1, said hydrolysis being conducted to an extent sufficient to provide a product having a dextrose equivalent value less than 50.

10. A method according to claim 1, said hydrolysis being conducted to an extent sufficient to provide a product having a dextrose equivalent value less than 20.

11. A method according to claim 1, said hydrolysis being conducted to an extent sufficient to provide a product having a dextrose equivalent value greater than or equal to 20.

12. A method according to claim 1, said hydrolysis being conducted to an extent sufficient to provide a product having a dextrose equivalent value in the range of 2 to 20.

13. A method according to claim 1, said hydrolysis being conducted in first and second stages, the first stage being conducted at a first temperature and the second stage being conducted at a second temperature, said second temperature being lower than said first temperature.

14. A method according to claim 1, said enzyme being fermented from Bacillus licheniformis.

15. A method according to claim 1, said calcium salt being present in an amount effective to process an initial calcium ion concentration of at least 40 ppm based on the weight of the starch.
